(19) United States
(12) Patent Application Publication
(10) Pub. No.: US 2019/0314261 A1
(43) Pub. Date: Oct. 17, 2019

(54) GLITTER AND THE USE THEREOF IN COSMETIC FORMULATIONS

(71) Applicant: Sigmund Lindner GmbH, Warmensteinach (DE)

(72) Inventor: Erwin Pschierer, Kastl (DE)

(73) Assignee: Sigmund Lindner GmbH, Warmensteinach (DE)

(21) Appl. No.: 16/380,565

(22) Filed: Apr. 10, 2019

(30) Foreign Application Priority Data
Apr. 12, 2018 (DE) 102018205562.9

(51) Int. Cl. A61K 8/73; A61K 8/26; A61K 8/02; A61Q 1/12; A61Q 1/10; A61Q 1/04; A61Q 19/00

(52) U.S. Cl. CPC A61K 8/731; A61K 8/26; A61K 8/0258; A61K 2800/624; A61Q 1/10; A61Q 1/04; A61Q 19/001; A61Q 1/12

(57) ABSTRACT

The present invention relates to glitter with improved lustre effect and improved skin feeling, and also glitter with improved surface coverage (covering power) and also the use thereof in cosmetic formulations, wherein the glitters comprise a film which comprises cellulose acetate.

The present invention relates to a glitter comprising a film which comprises cellulose acetate, to the use thereof in cosmetic products, and also to cosmetic products which comprise the glitter.

### PRIOR ART

Glitters have many applications for producing a sparkling surface effect and are used, inter alia and especially, in cosmetic items. In order to produce such glitters, foils or films made of plastic are used, which are cut into individual small particles of similar size by means of a cutting procedure. Currently, films made of polyethylene terephthalate are predominantly used for the production of glitter.

An example of a method for producing such glitters is disclosed in DE 102010001971 A1. Glitter which is coated on all sides is disclosed therein.

However, there is a further need for glitters having improved lustre effects. Furthermore, it is desirable to prepare glitters with an improved skin feeling, that is to say glitters which elicit a softer and more pleasant feeling on the skin when used in cosmetic products. Moreover, there is a need for glitters which have a better surface coverage (covering power).

The object of the invention is therefore that of providing glitters which have improved lustre effects and an improved skin feeling. Another object of the invention is that of providing glitters which have better surface coverage (covering power) and which can be used in cosmetic products due to their good compatibility.

### SUMMARY OF THE INVENTION

To date, films based on polyethylene terephthalate have predominantly been used for the production of glitter. Surprisingly, it was found that the object of the invention, that is to say providing glitter with improved lustre effect and improved skin feeling, and also glitter with improved surface coverage (covering power), could be solved by using films based on cellulose acetate in the production of glitter.

The present invention therefore relates to glitter comprising a film based on cellulose acetate. Furthermore, the invention relates to the use of glitter in cosmetic formulations.

In a first aspect, the present invention is directed towards glitter comprising a film which comprises cellulose acetate.

Also disclosed are the use of the inventive glitters in cosmetic products and also cosmetic products comprising the inventive glitters.

Further preferred developments of the present invention can be taken from the dependent claims and the following detailed description of the invention.

### DESCRIPTION OF THE FIGURES

The appended drawings are intended to illustrate embodiments of the present invention and aid further understanding thereof. In conjunction with the description, they serve to elucidate concepts and principles of the invention. Other embodiments and many of the advantages mentioned are evident in view of the drawings. The elements of the drawings are not necessarily shown in a manner true to scale relative to one another. Elements, features and components which are identical, functionally identical or act in identical ways are each given the same references in the figures of the drawings, unless indicated otherwise.

FIGS. 1 to 4 are schematic depictions of inventive glitter.

### DETAILED DESCRIPTION OF THE INVENTION

### Definitions

Unless otherwise defined, technical and scientific expressions used herein have the same meaning as that commonly understood by those skilled in the art in the field of the invention.

Stated amounts in the context of the present invention relate to wt %, unless otherwise indicated or unless obvious from the context. In the film which comprises cellulose acetate, the wt % proportions add up to 100 wt %.

Glitters are small particles with a wide variety of shapes. They especially have a size, for example a maximum diameter in a main direction of extent of the particle, of 0.02 mm to 7.0 mm, preferably 0.050 mm to 6.0 mm, for example 0.06 mm to 2.0 mm, e.g. 0.1 mm-0.5 mm, for example 100 μm to 200 μm. The glitter particles are not particularly restricted in terms of shape and may for example be in the form of platelets, needles, cuboids, etc., or may be punched in particular shapes, for example hexagons, squares, circles, ovals, stars, etc. According to specific embodiments, the glitters are configured to be flat, for example as platelets with a wide variety of shapes, e.g. also hexagonal, rectangular, square, star-shaped, round, oval, etc., wherein the thickness of the platelets may preferably be between 4 μm and 50 μm, for example between 5 μm and 45 μm, e.g. between 10 μm and 35 μm, by way of example between 14 μm and 23 μm, and/or the size, for example a maximum diameter in a main direction of extent of the glitter, may be from 0.02 mm to 7.0 mm, preferably 0.050 mm to 6.0 mm, for example 0.06 mm to 2.0 mm, e.g. 0.1 mm to 0.5 mm.

In a first aspect, the present invention relates to glitter comprising a film which comprises cellulose acetate. In this case, the cellulose acetate is not particularly restricted, for example in terms of the degree of substitution.

According to particular embodiments, the film comprises at least 60 wt % cellulose acetate, preferably more than 60 wt %, more preferably more than 70 wt %, yet more preferably more than 80 wt %, and especially preferably more than 90 wt %, for example more than 95 wt % or even more than 99 wt %, based on the weight of the film.

Alongside this, however, further additives and/or one or more further polymers, such as for example cellulose, modified cellulose, regenerated cellulose, starch, polylactic acid (PLA), polybutylene adipate terephthalate (PBAT), polybutylene succinate (PBS), thermoplastic starch (TPS), polyhydroxyalkanoate (PHA) etc., may also be present in the film in an amount of up to 40 wt %, preferably less than 40 wt %, more preferably less than 30 wt %, yet more preferably less than 20 wt %, and especially preferably less than 10 wt %, for example less than 5 wt %, or even less than 1 wt %, which are not particularly restricted.

For example, according to specific embodiments, the film may contain glycerol triacetate (triacetin) as additive. In this case, the proportion by mass of glycerol triacetate may preferably be less than 40 wt %, more preferably less than 30 wt %, yet more preferably less than 20 wt %, and especially preferably less than 10 wt %, for example less than 5 wt % or even less than 1 wt %, based on the weight of the film. The film may also not contain any glycerol triacetate. Alternatively or additionally, according to specific embodiments, the film may contain additives which may for example act as antiblocking agent and/or lubricant. These antiblocking agents and/or lubricants are not particularly restricted and may for example comprise amorphous silicas, fatty acid amides and/or talc. According to specific embodiments, the amount added of such antiblocking agents and/or lubricants may be 0-2.5 wt %, preferably 0-1.5 wt %, more preferably 0-1 wt % based on the weight of the film.

According to specific embodiments, the film essentially consists of cellulose acetate or just consists of cellulose acetate, except for unavoidable impurities.

According to specific embodiments, the glitter is further coated with a metal, preferably aluminium, silver, gold and/or copper, preferably aluminium. In this case, the coating may, according to specific embodiments, be produced on one side, on two opposing sides, on the whole film or in another manner.

According to specific embodiments, the film or a metal layer, for example an aluminium layer, is coated with a coating based on cellulose or modified cellulose, preferably based on cellulose nitrate, based on cellulose acetate butyrate, based on cellulose acetate propionate and/or based on cellulose acetate, and/or based on polyurethane, acrylate, styrene acrylate and/or epoxide. Here too, the coating may be present on one side, on two opposing sides, on the whole metal layer, for example aluminium layer, of the film and the metal layer, for example aluminium layer, or in another manner.

Special preference is given to coatings based on cellulose or modified cellulose, preferably based on cellulose nitrate, based on cellulose acetate butyrate, based on cellulose acetate propionate and/or based on cellulose acetate, especially based on cellulose acetate.

In addition, the inventive glitter may also comprise further colour-imparting and/or effect-imparting layers as are known to those skilled in the art and which can be suitably coated by those skilled in the art, for example from the gas phase or from liquid/solution.

According to specific embodiments, the film of the inventive glitter has a thickness of 5 μm or more, preferably of more than 10 μm, more preferably of more than 12 μm, and/or a thickness of 40 μm or less, for example less than 40 μm, preferably less than 30 μm, more preferably less than 28 μm and particularly preferably less than 25 μm, for example also less than 20 μm or even less than 15 μm.

Especially at these thicknesses of films comprising cellulose acetate, it is possible to achieve a further improved uniform distribution in the cosmetic formulation for the glitter according to the invention. As a result, after application to the skin, further improved lustre effects and a further improved surface coverage (covering power) are achieved.

According to specific embodiments, the film may additionally be transparent or essentially transparent, for example having a transmissivity for light in the visible range from 380 to 780 nm of 70%, or 80%, or 90%, or more. According to specific embodiments, the film therefore has a transmissivity for light in the wavelength range from 380 to 780 nm of at least 70%, preferably at least 80%, more preferably at least 90%.

According to further embodiments, the film may be coloured or colourless, but according to specific embodiments it is colourless. In addition, in specific embodiments, a colour-imparting layer may be applied to the coating with metal, preferably aluminium, or the film and the metal coating.

According to specific embodiments, the film has an E modulus of less than 3000 Nmm⁻², preferably 2000-2500 Nmm⁻², measured according to ASTM D883.

The comparatively low E modulus—corresponding to lower rigidity—has a positive effect on the soft and pleasant skin feel. Thus, also disclosed is a glitter comprising a film which has an E modulus of less than 3000 Nmm⁻², preferably 2000-2500 Nmm⁻², measured according to ASTM D883. Moreover, for this further, second aspect of the invention, the film is not restricted and may be any desired material, for example comprise or consist of cellulose derivatives such as cellulose acetate, polyethylene (PE), polypropylene (PP), PET, cellulose, regenerated cellulose, starch, PLA and/or further polymeric materials and/or mixtures thereof. This glitter of the second aspect may also further have the exemplary developments mentioned above and below, wherein in this case the material of the film is not restricted to one which comprises cellulose acetate. Also likewise disclosed, of course, are a cosmetic product comprising such a glitter of the second aspect, which is not particularly restricted and can be configured as stated below, and also the use of the glitter of the second aspect in a cosmetic product.

Overall, on account of the improved mechanical properties (E modulus, glidability) of the film used in the inventive glitter of the first aspect, a significantly improved skin feeling can be achieved with the inventive glitters of the first aspect, for example in a cosmetic product.

According to specific embodiments, in an inventive glitter the film may also be prepared with a hologram imprint, as is known in conventional glitter, as is known for example from U.S. Pat. No. 5,810,957 or EP2 163 381 A, wherein reference is made to both documents in respect of the hologram imprint. Surprisingly, the hologram imprint can also be produced on the film comprising cellulose acetate. In this case, for the preparation, customary methods can be used such as what are referred to as soft embossing and hard embossing.

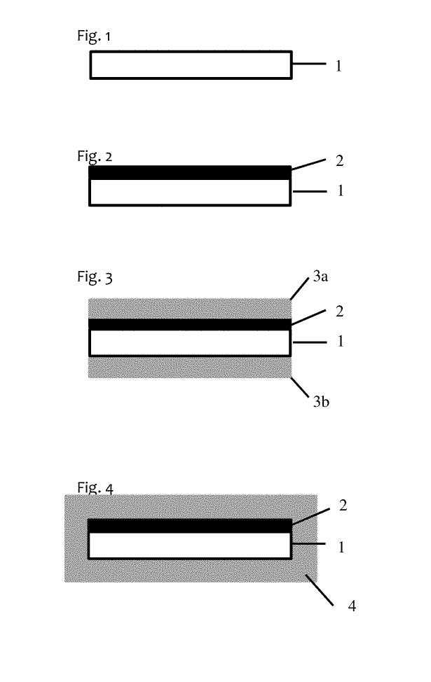

The invention will be explained in more detail below using exemplary embodiments of inventive glitters, especially of the first aspect, which are shown in FIGS. 1 to 4. Here, the figures show schematic sectional views through glitter with a construction according to the invention.

In this case, inventive glitters comprise for example glitter particles having a size of 100 μm to 200 μm and a thickness of 14 μm to 23 μm, but are not restricted thereto. According to a first exemplary embodiment which is illustrated in FIG. 1, transparent particles 1 essentially consisting of cellulose acetate can be obtained as films of the inventive glitters by cutting a cellulose acetate foil.

According to a further embodiment which is illustrated in FIG. 2, the particles 1 essentially consisting of cellulose acetate can be coated on one side with a metal layer, for example an aluminium layer 2. In this case, a coating made of aluminium can preferably be applied by vapour deposition under reduced pressure.

Moreover, the particles 1 essentially consisting of cellulose acetate and coated on one side with aluminium 2 can, in a further embodiment as shown in FIG. 3, additionally be coated evenly on two opposing sides with layers 3a, 3b, made for example of cellulose acetate.

Alternatively, the particles 1 essentially consisting of cellulose acetate and coated on one side with aluminium 2 can, in a further embodiment as shown in FIG. 4, be coated evenly on all sides with a layer 4 made for example of cellulose acetate.

In FIGS. 1 to 4, the film essentially consisting of cellulose acetate can also be coloured according to alternative refinements. In FIGS. 3 and 4, in addition, the layers of cellulose acetate 3a, 3b and/or 4 on the particles essentially consisting of cellulose acetate can also be coloured according to alternative developments.

The method for preparing the inventive glitters is not particularly restricted. Here, a film, especially comprising cellulose acetate, can be suitably cut to particles of a suitable size and optionally coated in a customary manner with metal, polymer, etc. Here, low tensile strength and low elongation at break, for example especially of cellulose acetate films, may have an effect on the processability during glitter production. In glitter production, the films are typically processed “on roll”. In this context, large tensile forces generally act on the films in the processing machines (e.g. film coating and/or glitter cutting machines). It is therefore necessary during processing to keep the tensile forces which arise as low as possible, in order to avoid tearing of the film in the processing machines. This may for example be achieved by a correspondingly configured web guidance in the processing machines and the corresponding design of the web-guiding transport rollers in order to avoid excessively high tensile forces on the film.

The application of different coatings is not particularly restricted and may for example be carried out in the gas phase and/or from solution.

A further aspect of the present invention relates to the use of inventive glitters in cosmetic products. The cosmetic product is not particularly restricted here. Cosmetic products comprise in this regard for example pastes, salves, creams, emulsions, solutions, lipstick, lip gloss, mascara, mousse, eyeshadow, eyeliner, powder, pressed powder, loose glitter powder, nail varnish, soaps, shampoo, sun protection compositions, lotions, aerosol sprays, etc., which may contain the glitter in customary amounts in the formulations.

Additionally disclosed is a cosmetic product which comprises inventive glitter. The cosmetic product in this regard is not particularly restricted, and it may for example be a paste, a salve, a cream, an emulsion, a solution, a lipstick, lip gloss, mascara, mousse, eyeshadow, eyeliner, powder, pressed powder, loose glitter powder, nail varnish, soap, shampoo, sun protection compositions, a lotion, an aerosol spray, etc., wherein the glitters may be contained in customary amounts in the cosmetic product, for example between 0.01 and 75 wt %, e.g. between 1 and 10 wt % based on the cosmetic product, or even up to 100 wt % in the case of powder and loose glitter powder. Alongside this, the cosmetic products may contain the customary constituents such as carriers, fillers, oils, waxes, fats, emulsifiers, antioxidants, film-formers, odorants and/or flavourings, stabilizers, solvents, surfactants, preservatives, thickeners, rheology additives, dyes, vitamins, buffer substances, cosmetic active agents, skin-active substances, e.g. skincare substances, UV filters, etc., which are all not particularly restricted. The cosmetic products may for example be of hydrophilic, hydrophobic and/or lipophilic nature. Corresponding constituents are known for example from DE 102005055576 A1, to which reference is for example made in respect of cosmetic formulations for the production of cosmetic products.

The above embodiments, developments and refinements may be combined with one another as desired, if practical. Further possible developments, refinements and implementations of the invention also encompass combinations—not explicitly mentioned—of features of the invention described above or below with reference to the exemplary embodiments. In particular, those skilled in the art will also add individual aspects as improvements or supplementations to the respective basic form of the present invention.

The invention will be described further in detail below with reference to various examples thereof. However, the invention is not restricted to these examples.

### EXAMPLES

### Example 1: Glitter

Inventive glitters according to FIG. 2 with a size of 100 μm to 200 μm and a thickness of 14 μm to 23 μm of a cellulose acetate film in the glitters were produced by coating a cellulose acetate film with aluminium by vapour deposition on one side and cutting to the predetermined size.

For comparison, glitters with films made of polylactic acid (PLA), regenerated cellulose and polyethylene terephthalate (PET) were produced by corresponding methods with the same size and coating.

In this case this gave different E moduli for the different materials, which are stated in table 1.

TABLE 1
E moduli of the films in the glitters in example 1

| | Cellulose | | Regenerated | |
|---|---|---|---|---|
| | acetate | PLA | cellulose | PET |
| E modulus | 2000-2500 | 3500-4000 | 3000-3500 | 3600-4500 |
| [Nmm⁻²] | | | | |
| ASTM D883 | | | | |

As well as the conventional PET (polyethylene terephthalate)-based glitters, glitters can also be obtained as a standard on the market which are based on biodegradable films. Corresponding glitters are known, produced from PLA (polylactose) or regenerated cellulose, and commercially available. In this regard, CA surprisingly has a significantly better degradability compared to PLA.

Besides this, compared to PLA and regenerated cellulose, the comparatively high temperature resistance of cellulose acetate (CA) enables the use in cosmetic products such as lipstick formulations, the production of which typically takes place at temperatures of up to 100° C.

Compared to PLA and regenerated cellulose, films based on CA also have improved chemical resistance, for example tested as resistance to ethanol. The good resistance profile of CA enables the use of the inventive glitters in the majority of cosmetic formulations.

Further commercially available, biodegradable films consist of polybutylene adipate terephthalate (PBAT), polybutylene succinate (PBS), thermoplastic starch (TPS) or polyhydroxyalkanoate (PHA). On account of material properties, these films are however not suitable for glitter production. PBAT, PBS and TPS form soft, elastic films which cannot be cut into glitter particles—this is related to the low glass transition temperatures (Tg) of these polymers. PBS has a glass transition temperature of −30° C. In comparison, the glass transition temperature of PLA is +50° C. TPS is also not resistant to hydrolysis. PHA-based films are cloudy-white and do not give high-lustre, reflective surfaces, in comparison to transparent films.

Thus, the outstanding utility of cellulose acetate films in inventive glitters, compared to those of the prior art, becomes apparent.

### Example 2: Application Examples in Cosmetic Products

In the following formulation examples, in each case one inventive glitter (glitter example according to FIG. 2 from example 1) and one corresponding glitter from the prior art (comparative glitter consisting of a polyethylene terephthalate film with Al coating of the same size from example 1) are comparatively assessed.

Different cosmetic products were produced in the customary manner with these glitters, with the constituents/ingredients and proportions (wt % based on the product) according to the respective data in tables 2, 3, 4 and 5.

### Product 1: Face Powder

TABLE 2
Composition of the face powder

| | Ingredient | % W/W |
|---|---|---|
| Phase A | Talc | 19.5 |
| | Mica | 39.3 |
| | Nylon 12 | 8.0 |
| | Silicon dioxide (silica) | 5.0 |
| | Zinc stearate | 6.0 |
| | Propylparaben | 0.2 |
| | CI 77491 (Iron Oxide Red) | 0.3 |
| | CI 77492 (Iron Oxide Yellow) | 0.7 |
| Phase B | Tridecyl stearate (and) tridecyl trimellitate | 2.0 |
| | (and) dipentaerythrityl | |
| | hexacaprylate/hexacaprate | |
| Phase C | Glitter | 19.0 |
| | Sum | 100.0 |

### Production:

1. Mix ingredients of phase A
2. Mix phase B and phase C into phase A

The face powder with the inventive glitter is more lustrous and has a softer skin feel than the face powder with the comparative glitter.

### Product 2: Eyeshadow (Pressed)

TABLE 3
Composition of the eyeshadow

| | Ingredient | % W/W |
|---|---|---|
| Phase A | Talc | 32.9 |
| | Mica | 21.9 |
| | Boron nitride | 2.0 |
| | Silicon dioxide (silica) | 5.0 |
| | Magnesium stearate | 3.0 |
| | Propylparaben | 0.2 |
| Phase B | Dimethicone | 5.0 |
| | METHOXY PEG-17/METHOXY | 5.0 |
| | PEG-11/HDI CROSSPOLYMER | |
| | Glitter | 25.0 |
| | Sum | 100.0 |

### Production:

1. Mix ingredients of phase A
2. Mix ingredients of phase B
3. Homogeneously mix phase A and phase B

### 4. Press for 30 min at 150 bar

The eyeshadow with the inventive glitter is more lustrous and has a better covering power than the eyeshadow with the comparative glitter.

### Product 3: Lip Gloss

TABLE 4
Composition of the lip gloss

| | Ingredient | % W/W |
|---|---|---|
| Phase A | Paraffinum Liquidum | 42.7 |
| | Polyisobutene | 37.4 |
| | Triisodecyltrimellitate | 4.0 |
| | Mineral oil (and) ethylene/propylene/styrene | 3.0 |
| | copolymer & butylene/ethylene/styrene | |
| | copolymer | |
| | Silica Dimethyl Silylate | 2.0 |
| | Ethylhexylpalmitate | 2.0 |
| | Hydrogenated polyisobutene | 2.0 |
| | Polyethylene | 1.5 |
| | Sorbitan sesquiisostearate | 1.0 |
| | Mineral wax (Cera Microcristallina) | 0.5 |
| | Propylparaben | 0.2 |
| Phase B | Glitter | 3.7 |
| | Sum | 100.0 |

### Production:

1. Homogeneously mix ingredients of phase A
2. Stir phase B into phase A

The lip gloss with the inventive glitter has a softer skin feel than the lip gloss with the comparative glitter.

### Product 4: Eyeliner

TABLE 5
Composition of the eyeliner

| | Ingredient | % W/W |
|---|---|---|
| Phase A | Water | 73.5 |
| | Xanthan (Xanthan Gum) | 0.9 |
| | Glitter | 2.5 |
| Phase B | Propylene glycol | 2.0 |
| | Phenoxyethanol (and) ethylhexylglycerin | 0.8 |
| | Polyurethane-35 | 19.5 |
| | Laureth-4 | 0.8 |
| | Sum | 100.0 |

### Production:

1. Homogeneously mix ingredients of phase A
2. Add ingredients of phase B sequentially and mix together

The eyeliner with the inventive glitter has a more uniform distribution of the glitter particles and thereby exhibits a better lustrous effect than the eyeliner with the comparative glitter.

1. A glitter comprising a film which comprises cellulose acetate.

2. The glitter of claim 1, wherein the film comprises at least 60 wt % cellulose acetate based on the weight of the film.

3. The glitter of claim 1, wherein the film essentially consists of cellulose acetate.

4. The glitter of claim 1, wherein the glitter is further coated with a metal layer.

5. The glitter of claim 4, wherein the film or the metal layer is coated with a coating based on cellulose or modified cellulose, cellulose acetate butyrate, cellulose acetate propionate, cellulose acetate, polyurethane, acrylate, styrene acrylate, and/or epoxide.

6. The glitter of claim 1, wherein the film has a thickness of 5 μm or more.

7. The glitter of claim 1, wherein the film has a transmissivity for light in the wavelength range from 380 to 780 nm of at least 70%.

8. The glitter of claim 1, wherein the film has an E modulus of less than 3000 Nmm⁻² measured according to ASTM D883.

9. Use of the glitter of claim 1 in a cosmetic product.

10. A cosmetic product comprising the glitter of claim 1.

11. The glitter of claim 4, wherein the metal is aluminum.

12. The glitter of claim 5, wherein the film or the metal layer is coated with a coating based on cellulose nitrate.

13. The glitter of claim 1, wherein the film has a thickness of 40 μm or less.

14. The glitter of claim 1, wherein the film has a thickness of 5 μm to 40 μm.

15. The glitter of claim 8, wherein the film has an E modulus of 2000-2500 Nmm⁻² measured according to ASTM D883.
